(19) United States
(12) Patent Application Publication
(10) Pub. No.: US 2015/0080780 A1
(43) Pub. Date: Mar. 19, 2015

(54) CONTOURED JOINT BRACE WITH SUPPORT STIFFNESS INSERT

(71) Applicant: Stacia Pache, Redmond, WA (US)

(72) Inventor: Stacia Pache, Redmond, WA (US)

(21) Appl. No.: 14/029,792

(22) Filed: Sep. 17, 2013

(51) Int. Cl. A61F 5/01

(52) U.S. Cl. CPC A61F 5/0123
USPC 602/26

(57) ABSTRACT

A method and an article of manufacture are disclosed for supporting joints including a contoured brace having a Support Stiffness Insert (SSI) configured to be inserted into a cavity within the joint brace. In various embodiments, the contours of the brace fit the curves of the body to increase support, mobility and comfort. The SSI has a asymmetrical shape with a curved surface facing the body joint and a flat surface facing the opposite direction. The SSI is made of flexible materials such as rubber, gel, neoprene, and the like, while in other various embodiments, the SSI comprises a fluid sack with adjustable volume, pressure, stiffness, and shape of the joint brace. The joint brace may include a Decorative Pattern Insert (DPI) inserted into a transparent window to change the decorative appearance of the joint brace or to reduce its visibility by using a DPI with natural skin tones.

### TECHNICAL FIELD

This application relates generally to medical devices. More specifically, this application relates to a bracing apparatus for supporting joints, such as the knee.

### BRIEF DESCRIPTION OF THE DRAWINGS

The drawings, when considered in connection with the following description, are presented for the purpose of facilitating an understanding of the subject matter sought to be protected.

FIG. 1 shows an example exercise activity in which a knee brace is used to support the knee joints;

FIG. 2 shows an example size-adjustable knee brace with a support stiffness insert;

FIG. 3A shows an example side view of a knee joint using the size-adjustable knee brace of FIG. 2;

FIG. 3B shows an example side view of a knee joint using the size-adjustable knee brace of FIG. 2;

FIG. 4 shows an example exploded view of the size-adjustable knee brace with a support stiffness insert, depicting the insertion of the insert into the brace;

FIG. 5 shows an example exploded view of the size-adjustable knee brace with a decorative pattern insert, depicting the insertion of the insert into the brace; and

FIG. 6 shows an example size-adjustable knee brace with an adjustable stiffness control.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description references using a the knee joint, it will be appreciated that the disclosure may be applicable to other body joints, such as the elbow joint, the shoulder joint, the ankle joint, and the like.

Briefly described, a method and an article of manufacture are disclosed for supporting joints including a contoured brace having a Support Stiffness Insert (SSI) configured to be inserted into an opening deployed within the joint brace to locally and precisely support the tendons and ligaments surrounding the joints. In various embodiments, the contours of the contoured brace are configured to precisely sit on and fit the curves of the body around the joints to increase support at the precise points on the joint where support is needed most, while also increasing mobility and comfort. In some embodiments, the SSI has a asymmetrical shape with a curved surface facing the body joint and a flat surface facing the opposite direction. In various embodiments, the SSI is made of flexible and firm materials such as rubber, gel, neoprene, and the like, while in other various embodiments, the SSI comprises a fluid sack with adjustable volume and pressure to adjust the stiffness and shape of the joint brace. In other embodiments, the joint brace includes a Decorative Pattern Insert (DPI) that may be inserted into a transparent window within the joint brace to change the decorative appearance of the joint brace or to reduce its visibility by using a DPI with natural skin tones.

With the increased health-consciousness and the extensive promotion of exercise for fitness and appearance, more people are becoming physically active. The increased physical activity results in increased sports-related injuries, such as joint problems. Joint problems often result from sports and exercises including impact forces, such as running and jumping, which are common in many sports. Other activities, such as skiing, contribute to joint injuries due to twisting motion during weight-bearing motions. A ubiquitous, simple, and cost effective device for protecting, supporting, and/or relieving pain from injured or stressed/strained joints is a joint brace. Most joint braces are elastic to provide firm support. A few braces are non-elastic strips, and like belts, work based on fixed tension created by pulling and then fastening the end of the brace strip using hook-and-loop fasteners like VELCRO®, buckles, and the like.

Most existing joint braces suffer from a number of shortcomings. Fitting body contours is difficult to achieve with one or a few sizes, especially when the width or other dimension of the brace is substantially fixed. This way, the brace material may pile up on the side of the joint that bends, causing discomfort and limiting the range of motion. Such configurations may also create unintended distribution of pressure or force of the brace around the joint in a manner which is not useful or desirable. For example, a uniform sleeve-like brace in the form of a hollow cylinder may create pressure points on places around the joint by getting stretched/pulled, which does not necessarily need the extra pressure, while reducing or eliminating pressure on points on the joint that does need the extra support by getting slacked. An extension of this problem is that traditionally, many of the joint support braces have been originally designed for men, and thus, may not fit women's anatomy as well, further exacerbating the problem of fit.

Another problem is that the firmness of the support and its distribution are usually not adjustable. So, if for some activity more support is needed, a different brace may have to be used. Even exchanging braces may not provide an ideal solution because the amount of support needed may vary by type of exercise, duration of the exercise, and the condition of the body.

Another problem is the appearance of the brace, which may not be esthetically pleasing.

Hence, a brace having a contour for good fit around the joint, having adjustable size and firmness, and having a changeable appearance is highly desirable.

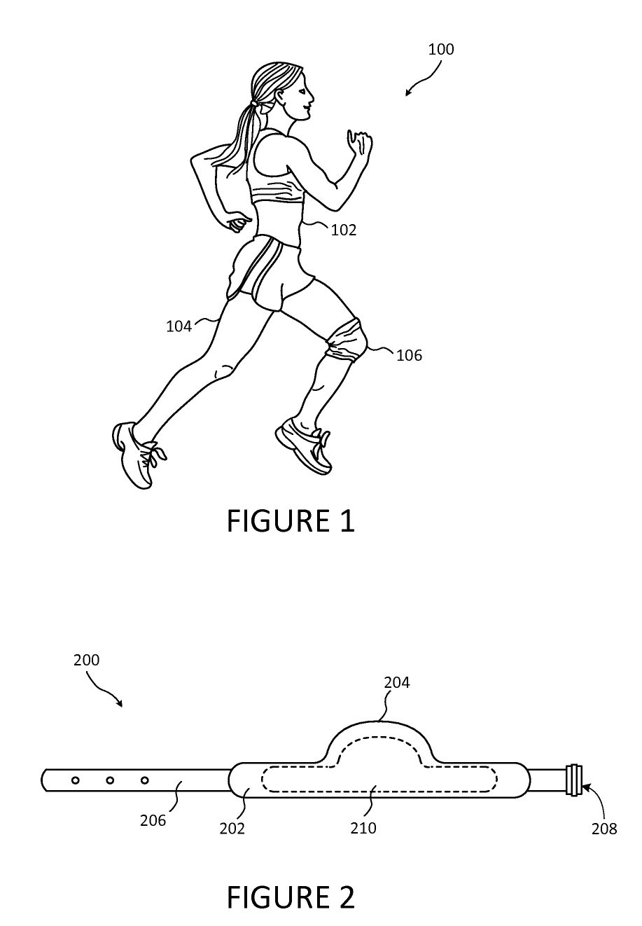

FIG. 1 shows an example exercise activity in which a knee brace is used to support the knee joints. An exercise activity 100 typically includes a sportsperson 102 using major muscle groups such as legs 104 and joint support braces 106. Most joint support braces are hollow elastic tubes, which surround the joint and give it elastic support. The level of support depends on the size of the brace relative to the limb and is not adjustable once made. Elastic material also age with time and lose some of its elasticity and thus the level of support provided. Many physical activities such as running, walking, hiking, basketball, soccer, football, baseball, ski, and the like impart impact force or cause stress and strain to the joints, especially the knee joint. As such, joint support braces are highly useful in reducing such stress/strain, relive pain, and provide comfort.

FIG. 2 shows an example size-adjustable knee brace with a support stiffness insert. In various embodiments, a contoured, size-adjustable, support-adjustable knee brace 200 including support pad 202, support flap 204, adjustable band 206, buckle 208, and a cavity to hold Support Stiffness Insert (SSI) 210.

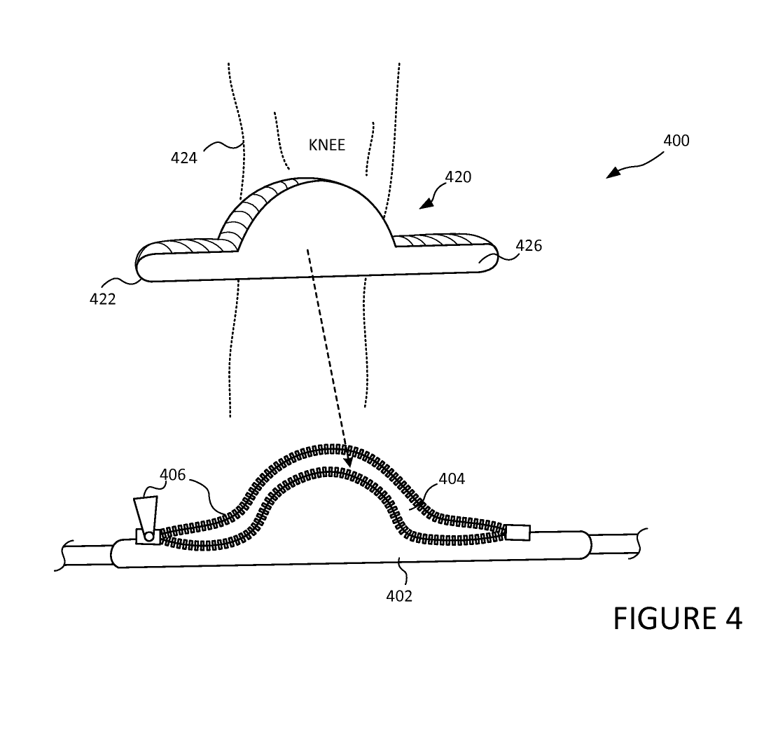

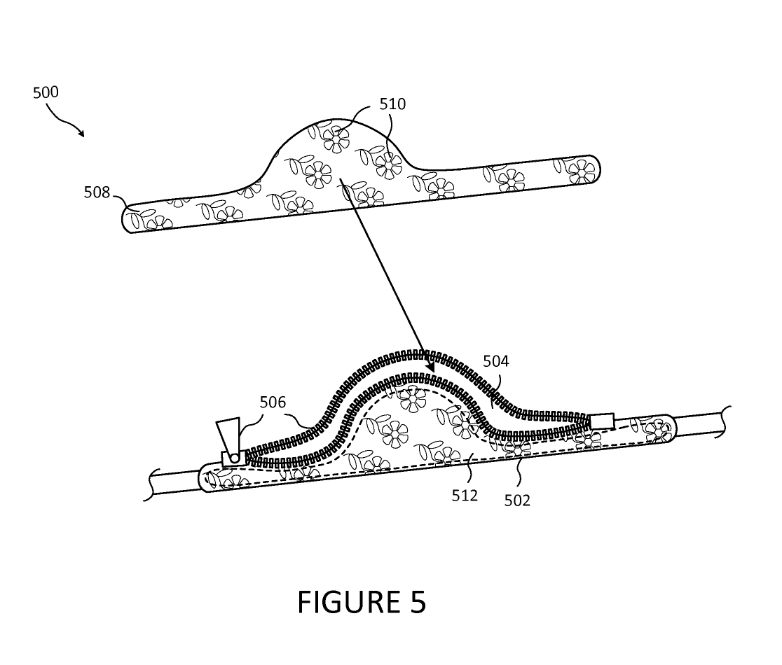

In various embodiments, the contoured knee brace 200 includes one or more cavities or pockets accessed through an opening in the support pad 202. In some embodiments, the opening may be controlled by a zipper, while in other embodiments, a hook-and-loop fastener, such as VELCRO® may be used to open and close the opening for access to the cavity, as further described with respect to FIGS. 4 and 5 below. In other embodiments, the cavity may have no device for closing it, such as a zipper, and remain open. In these embodiments, the cavity, or pocket may have firm or semi-rigid walls to maintain its shape and hold the SSI in place. In some embodiments, the cavity holds the SSI 210, while in other embodiments, the same or a second cavity may be transparent to hold a decorative pattern insert to change the appearance of the brace. In still other embodiments, the SSI may have a decorative pattern printed thereon.

In some embodiments, the support pad 202 may be made of a transparent material or a sturdy mesh to allow seeing the decorative pattern insert from outside. The decorative pattern insert may include various patterns, solid colors, skin-tone colors, and the like. The skin-tone colors make the support pad less visible and noticeable as may be preferred by the user.

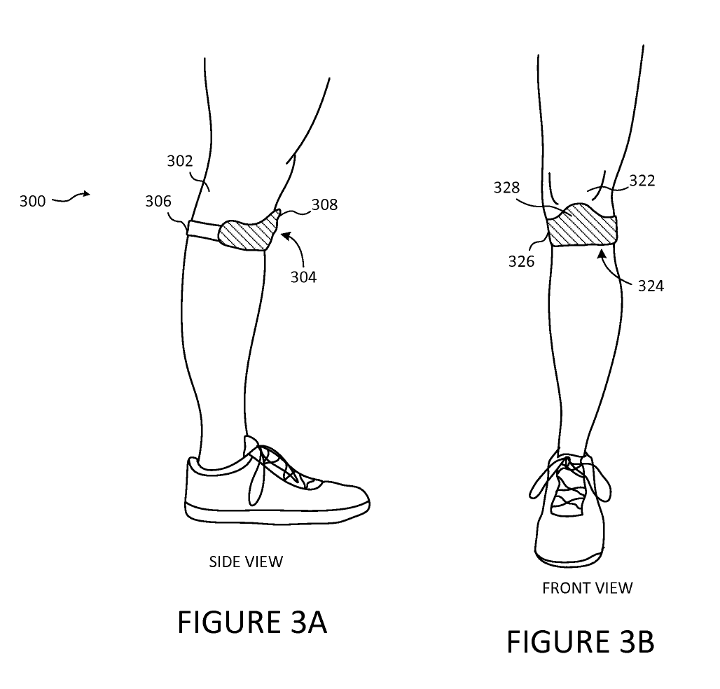

In various embodiments, support pad 202 and support flap 204 may include curves and be contoured to fit the curves of the body better depending on the joint. For example, the elbow joint may need a different contour than the knee or ankle joints. FIG. 2 shows a contour of the flap 204 suitable for providing extra support for the knee cap and tendons of the knee joint, as further described with respect to FIGS. 3A and 3B below.

In various embodiments, the SSI may be made from a flexible, soft, and supple material such as soft rubber, neoprene, dense foam, and the like to gently but firmly press against the joint to physically support the joint in comfort. In other embodiments, the SSI may be in the form of a fluid filled sac filled with water, oil, air, or other suitable fluid. The fluid pressure may be adjustable in some embodiments. In some embodiments, multiple SSI may be provided each having different thickness, firmness, material, and the like to allow the user to interchange the SSI best suited to his physical activity. In some embodiments, the SSI may be made asymmetrically to better distribute the support force on particular points on the joint, as further described below with respect to FIG. 4.

In various embodiments, adjustable band 206 may be a belt-and-buckle type of strap, a hook-and-loop type of fastener, adjustable elastic band, or any other type of fastener which may be used to adjust the size around the limb or body part. The same band may be used to control and adjust the compressive force exerted by the brace to support the joint. In various embodiments, the adjustable band 206 may be rubberized, be made of rubber, other non-slip material, or otherwise textured to prevent slippage during physical activity. In various embodiments, the band is slimmer and/or narrower than the support pad 202 to allow a more comfortable fit behind the knee or the bending side of other joints.

FIG. 3A shows an example side view of a knee joint using the size-adjustable knee brace of FIG. 2. In various embodiments, brace arrangement 300 includes support brace 304 having a support flap 308 and adjustable strap 306 around user's knee 302.

In various embodiments, strap 306 is adjusted to the desired tightness level around the knee, while positioning the support flap 308 on the front of the knee where the most support is needed to relieve pain or stress. In various embodiments, the amount of support the support flap 308 provides depends on the SSI's size and firmness. The SSI may be changed to allow a suitable SSI to be used as appropriate for the activity or preferred by the user.

FIG. 3B shows an example side view of a knee joint using the size-adjustable knee brace of FIG. 2. In various embodiments, joint brace 324 includes support pad 326 and support flap 328 to support the user's knee 322.

In various embodiments, the joint brace 324 may be moved up and down the user's knee and its size and firmness may be adjusted using the strap holding the brace in place. The support flap 328 provides extra support for the knee cap while the brace itself may be wrapped below the knee joint for comfort and ease of bending the knee and other movements. The firmness of the brace and/or flap may further be adjusted by using different SSI intended for different activities or preferences of the user.

FIG. 4 shows an example exploded view of the size-adjustable knee brace with a support stiffness insert, depicting the insertion of the insert into the brace. In various embodiments, joint brace assembly 400 includes support pad 402, cavity 404 accessed through zipper 406, and Support Stiffness Insert (SSI) 420. SSI 420 includes flat surface 426, and a semi-circular or curved cross section 422 of a curved surface, opposite the flat surface 426 of SSI 420, facing knee 424.

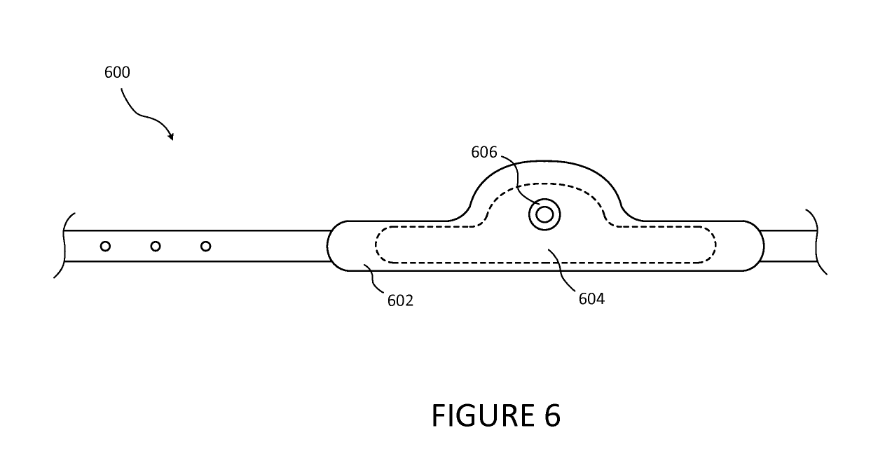

In various embodiments, the SSI may be made from a soft and pliable material such as soft rubber, synthetic rubber, neoprene, dense foam, natural sponge, silicone sponge, or a combination thereof, and the like to evenly distribute force around the joint to physically support the joint in comfort and avoid pressure points. In other embodiments, the SSI may be in the form of a fluid filled sac filled with water, oil, air, gel or other suitable fluid. The fluid pressure may be adjustable in some embodiments, as further described with respect to FIG. 6 below. In some embodiments, multiple SSI may be provided each having different thickness, firmness, material, and the like to allow the user to interchange the SSI best suited to his physical activity, fatigue, weather, or other conditions. In still other embodiments, the SSI may be hollow, combining the pliability of the solid material itself with the flexibility of the hollow chamber inside, like an under-inflated ball made of soft material. In other embodiments, the SSI may be a hybrid or laminated combination of material, fluid sac, and hollow chambers to provide different mechanical properties as needed at different locations around the joint.

In some embodiments, the SSI may have an asymmetrical shape and/or cross section to better focus the support force on particular points on the joint using softly curved surfaces, while avoiding the creation of sharp pressure points. In some embodiments, a set of SSI may be provided with the support brace, each SSI having a different shape, thickness, material, color, and other characteristics, each suited to different situations and uses.

In various embodiments, the SSI may have a flat surface facing away from the user's joint, and a curved surface facing towards the joint. The curved surface may be fixed or variable. A fixed curve has a fixed or uniform curvature, such as a semi-circle, while a variable curve has a variable, non-uniform curvature which may be more pronounced in one area and less pronounced in another to better fit the body contour around the joint. In various embodiments, the contour or curvature of the SSI may be custom made to fit a particular person's body contours.

In various embodiments, the SSI is inserted inside the cavity 404 of the support pad 402 via a zipped opening. In some embodiments, the zipper may be replaced with loop-and-hook fastener, while in other embodiments, a reusable adhesive strip may be used to open and close the cavity. Those skilled in the art will appreciate that other techniques for securing the cavity may be employed without departing from the spirit of the present disclosure. For example, small buttons or snaps may be used open and close the cavity.

FIG. 5 shows an example exploded view of the size-adjustable knee brace with a decorative pattern insert, depicting the insertion of the insert into the brace. In various embodiments, knee brace 500 may include support pad 502, transparent window 512, and zipper 506 for opening and closing cavity 504. In various embodiments, Decorative Pattern Insert (DPI) 508 may include pattern 510.

In various embodiments, The DPI may include various patterns, solid colors, skin-tone colors, and the like, which may be seen through the transparent window 512. The skin-tone colors make the support pad less visible and noticeable as may be preferred by the user in non-sporting circumstances such as for female users wearing a dress or skirt when going to work.

In various embodiments, the DPI may be integrated with SSI and be inserted as one piece into the cavity of the joint support brace. For example, the DPI may be a layer laminated on the flat surface 426 of the SSI shown in FIG. 4. In other embodiments, the DPI may simply be inserted into the same cavity as the SSI and cover the SSI as viewed through the transparent window. In other embodiments, two cavities may be provided, one for the SSI and one for the DPI so that each insert can be easily exchanged independent of the other insert.

FIG. 6 shows an example size-adjustable knee brace with an adjustable stiffness control. In various embodiments, knee brace 600 includes support pad 602, fluid sac 604, and stiffness adjustment valve 606.

In various embodiments, the SSI fluid sac 604 holds suitable fluids such as air, water, oil, gel, and the like. In some embodiments, the fluid control valve or stiffness adjustment valve 606 is a combination air valve and small hand or squeeze pump to adjust the volume and pressure of the air held within the SSI fluid sac. This way, the user has the ability to adjust the pressure, support, and stiffness of the SSI to suit her as she is wearing the support brace. In some embodiments, the control valve is attached to or integrated directly with the SSI fluid sac, while in other embodiments, the control valve is coupled with the support pad 602 which is in turn coupled with the SSI fluid sac, for example, via a small mechanical valve interface. In various embodiments, the SSI fluid sac may be an integral non-removable part of the support pad, while in other embodiments, the SSI may be a separate component which is inserted into a cavity within the support pad, as described above with respect to at least FIGS. 2 and 4.

In some embodiments, the fluid sac may include multiple separate chambers, each chamber having a different and/or separate fluid control valve. For example, fluid sac 604 may include a center chamber and two side chambers, each chamber controlled separately to provide the desired stiffness and support for the respective area of the joint closest to that chamber.

In other embodiments, the multiple chambers may be configurable. For example, multiple chambers may be connected together or be separated under user control to allow control of one or more chambers using a single stiffness control valve. In some embodiments, a sealable passageway between two adjacent chambers may be opened or closed, for example, using re-sealable plastic zip channels similar to Ziploc® or other similar techniques, to configure the stiffness control mechanism for the chambers. This arrangement allows the user to control the shape and size of the SSI independent of the stiffness. When two adjacent chambers are separated, then the fluid control valve only controls one chamber and can increase the fluid pressure/volume in the one chamber limiting the size of the one chamber and the shape of the SSI. When the two chambers are connected, the same control valve pressurizes both chambers and for the same pressure a larger volume results, again affecting the shape and increasing the size and thickness of the SSI.

In various embodiments, when the fluid in the fluid sac 604 is a liquid such as water or oil, an external pump may be used to adjust the fluid pressure. As the amount and/or the pressure of the fluid is adjusted, the shape of the sac, and thus, the contour of the SSI also change. This allows the control of multiple characteristics of the SSI including shape, curvature, stiffness, size/thickness, and the like. In some embodiments, one or more of these SSI characteristics may be controlled independently of the others, as described above with respect to multiple chamber configurations.

Changes can be made to the claimed invention in light of the above Detailed Description. While the above description details certain embodiments of the invention and describes the best mode contemplated, no matter how detailed the above appears in text, the claimed invention can be practiced in many ways. Details of the system may vary considerably in its implementation details, while still being encompassed by the claimed invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the claimed invention to the specific embodiments disclosed in the specification, unless the above Detailed Description section explicitly defines such terms. Accordingly, the actual scope of the claimed invention encompasses not only the disclosed embodiments, but also all equivalent ways of practicing or implementing the claimed invention.

The above specification, examples, and data provide a complete description of the manufacture and use of the composition of the invention. Since many embodiments of the invention can be made without departing from the spirit and scope of the invention, the invention resides in the claims hereinafter appended. It is further understood that this disclosure is not limited to the disclosed embodiments, but is intended to cover various arrangements included within the spirit and scope of the broadest interpretation so as to encompass all such modifications and equivalent arrangements.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. An apparatus for joint support, the apparatus comprising:
a support pad with a cavity deployed therein;
an adjustable strap coupled with the support pad; and
a Support Stiffness Insert (SSI) made from a flexible material configured to be inserted into the cavity of the support pad.

2. The apparatus of claim 1, further comprising a decorative pattern insert configured to be inserted into the cavity of the support pad.

3. The apparatus of claim 1, wherein the support pad includes a support flap.

4. The apparatus of claim 1, wherein the cavity of the support pat is opened and closed using a fastener.

5. The apparatus of claim 1, wherein the SSI is made from at least one of soft rubber, synthetic rubber, neoprene, dense foam, natural sponge, silicone sponge, or a combination thereof.

6. The apparatus of claim 1, wherein the SSI comprises a fluid sac.

7. The apparatus of claim 6, wherein the fluid sac comprises multiple separate chambers.

8. The apparatus of claim 6, wherein a pressure of a fluid in the fluid sac is controlled using a control valve.

9. The apparatus of claim 1, wherein the SSI has a flat surface and curved surface, wherein the curved surface faces towards a joint.

10. A method of treatment of a strained joint, the method comprising:
using a support pad with a cavity deployed therein;
wrapping an adjustable strap coupled with the support pad around the strained joint; and
inserting a Support Stiffness Insert (SSI) made from a flexible material into the cavity of the support pad.

11. The method of claim 10, further comprising inserting a decorative pattern insert into the cavity of the support pad.

12. The method of claim 10, further comprising positioning a support flap, integrated with the support pad, on the strained joint.

13. The method of claim 10, wherein the support pad and the SSI include a contour configured to closely fit the strained joint.

14. The method of claim 10, wherein the SSI is made from at least one of soft rubber, synthetic rubber, neoprene, dense foam, natural sponge, silicone sponge, or a combination thereof.

15. The method of claim 10, wherein the SSI comprises a fluid sac containing a fluid.

16. The method of claim 15, further comprising using a control valve coupled with the SSI to control the pressure of the fluid in the fluid sac.

17. An apparatus for knee joint support, the apparatus comprising:
a support pad with a cavity deployed therein and a contoured flap configured to provide additional support to a knee cap of the knee joint;
an adjustable strap coupled with the support pad; and
a Support Stiffness Insert (SSI) made from a flexible material configured to be inserted into the cavity of the support pad.

18. The apparatus of claim 17, wherein adjustable strap is narrower than the support pad.

19. The apparatus of claim 17, wherein the SSI has a flat surface and curved surface, wherein the curved surface faces towards a joint.

20. The apparatus of claim 17, wherein the SSI comprises a fluid sac containing a fluid.
